(19) United States
(12) Patent Application Publication
(10) Pub. No.: US 2022/0406159 A1
(43) Pub. Date: Dec. 22, 2022

(54) Fall Risk Assessment System

(71) Applicant: Hitachi, Ltd, Chiyoda-ku, Tokyo (JP)

(72) Inventors: Yuan LI, Tokyo (JP); Pan ZHANG, Beijing (CN)

(21) Appl. No.: 17/640,191

(22) PCT Filed: Mar. 19, 2020
(86) PCT No.: PCT/JP2020/012173

§ 371 Date: Mar. 3, 2022

(51) Int. Cl. G08B 21/04; G08B 29/18; G06V 40/20; G06V 40/16; G06T 7/285

(52) U.S. Cl. CPC G08B 21/043; G08B 21/0476; G08B 29/185; G06V 40/25; G06V 40/172; G06T 7/285; G06T 2207/10012; G06T 2207/30196

(57) ABSTRACT

The purpose of the present invention is to provide a fall risk evaluation system whereby risk of falling of an elderly person or other person to be managed can be easily evaluated on the basis of a captured image of daily life, instead of by a physical therapist, etc. To achieve this purpose, the present invention is a fall risk evaluation system comprising a stereo camera and a fall risk evaluation device, the fall risk evaluation device being provided with: a person authentication unit for authenticating a person to be managed who has been imaged by the stereo camera; a person tracking unit for tracking the person to be managed who is authenticated by the person authentication unit; an action extraction unit for extracting walking by the person to be managed; a feature value calculation unit for calculating a feature value of the walking extracted by the action extraction unit; an integration unit for generating integrated data obtained by integrating the outputs of the person authentication unit, the person tracking unit, the action extraction unit, and the feature value calculation unit; a fall index calculation unit for calculating a fall index value of the person to be managed, on the basis of a plurality of integrated data generated by the integration unit; and a fall risk evaluation unit for comparing the fall index value calculated by the fall index calculation unit and a threshold value to evaluate the risk of falling of the person to be managed.

### TECHNICAL FIELD

The present invention relates to a fall risk assessment system which assesses the fall risk of a target person to be managed such as an elderly person, based on images taken in daily life.

### BACKGROUND ART

Various long-term care services such as home care services, home medical services, homes for the elderly with long-term care, long-term care insurance facilities, medical treatment type facilities, group homes, and day care have been provided to elderly people requiring long-term care, etc. In these long-term care services, many experts work together to provide various services such as health checks, health management, and life support to the elderly. For example, a physiotherapist routinely visually assesses each person's physical condition and advises on physical exercise which suits the physical condition in order to maintain the body function of the elderly requiring long-term care.

On the other hand, in the endowment care business in recent years, the range of services to be provided is expanding even to elderly people who do not need long-term care and who need support, and healthy elderly people. However, the rapid increase in needs of the endowment care business has not caught up with the training of experts such as physiotherapists who provide long-term care and support services, and hence the lack of resources for the long-term care and support services has become a social problem.

Therefore, in order to improve this resource shortage, long-term care and support services using IoT devices and artificial intelligence are becoming widespread. For example, Patent Literature 1 and Patent Literature 2 have been proposed as a technique for detecting or predicting a fall in an elderly person on behalf of a physiotherapist, a caregiver, or the like.

The abstract of Patent Literature 1 describes, as a solving means for “providing a detection device which detects an abnormal state such as a fall or falling down of an observed person in real time from each captured image and removes the effects of background images or noise to improve the accuracy of detection.”, that “the detection device calculates the motion vector of each block of the image of the video data 41, and extracts the block in which the magnitude of the motion vector exceeds a fixed value. The detection device groups adjacent blocks together. The detection device calculates the feature amounts such as the average vector, the dispersion, and the rotation direction of the operation blocks included in the blocks in order from the blocks large in area, for example. The detection device detects, based on the feature amount of each group that the observed person is in an abnormal state such as a fall or falling down, and notifies the result of its detection to an external device or the like. The detection device corrects the deviation of the angle in the shooting direction, based on thinning processing of pixels in the horizontal direction with respect to the image, and the acceleration of a camera, to improve the accuracy of detection.”

Further, the abstract of Patent Literature 2 describes, as a solving means for “making it possible to accurately predict the occurrence of a fall from sentences contained in an electronic medical record”, that “there are provided a learning data input unit 10 which inputs m sentences included in an electronic medical record of a patient, a similarity index value calculation unit 100 which extracts n words from the m sentences and calculates a similarity index value which reflects the relationship between the m sentences and n words, a classification model generation unit 14 which generates a classification model for classifying the m sentences into a plurality of events, based on a sentence index value group consisting of n similarity index values for one sentence, and a risky behavior prediction unit 21 which applies the similarity index value calculated by the similarity index value calculation unit 100 from a sentence input by a prediction data input unit 20 to the classification model to thereby predict the possibility of the occurrence of a fall from the sentence to be predicted, whereby a highly accurate classification model is generated using a similarity index value indicating which word contributes to which sentence to what extent.”

### CITATION LIST

### Patent Literature

PTL 1: Japanese Unexamined Patent Application Publication No. 2015-100031

PTL 2: Japanese Unexamined Patent Application Publication No. 2019-194807

### SUMMARY OF INVENTION

### Technical Problem

Patent Literature 1 is for detecting an abnormality such as a fall of an observed person in real time, based on a feature amount of the observed person calculated from a photographed image. This is however not for analyzing the risk of falling of the observed person or predicting the falling in advance. Therefore, a problem arises in that even if the technology of Patent Literature 1 is applied to daily care/support for the elderly, etc., it is not possible to grasp deterioration in walking function from a change in the fall risk of a certain elderly person, or provide in advance fall preventive measures to the elderly with increased risk of falls.

Further, Patent Literature 2 is for predicting a patient's fall in advance, but since it is for predicting the occurrence of a fall by analyzing sentences included in the electronic medical record, the recording of the electronic medical record is essential for each patient. Therefore, a problem arises in that in order to apply to daily care and support for the elderly or the like, detailed text data equivalent to an electronic medical record must be created for each elderly person, so that the burden on a caregiver becomes very large.

Therefore, the present invention aims to provide a fall risk assessment system which can easily assess the fall risk of a target person to be managed such as an elderly person on behalf of a physiotherapist or the like on the basis of photographed images of daily life taken by a stereo camera.

### Solution to Problem

Therefore, the fall risk assessment system of the present invention is a system which is equipped with a stereo camera which photographs a target person to be managed and outputs a two-dimensional image and three-dimensional information, and a fall risk assessment device which assesses the fall risk of the managed target person, and in which the fall risk assessment device includes a person authentication unit which authenticates the managed target person photographed by the stereo camera, a person tracking unit which tracks the managed target person authenticated by the person authentication unit, a behavior extraction unit which extracts the walking of the managed target person, a feature amount calculation unit which calculates a feature amount of the walking extracted by the behavior extraction unit, an integration unit which generates integrated data which integrates the outputs of the person authentication unit, the person tracking unit, the behavior extraction unit, and the feature amount calculation unit, a fall index calculation unit which calculates a fall index value of the managed target person, based on a plurality of the integrated data generated by the integration unit, and a fall risk assessment unit which compares the fall index value calculated by the fall index calculation unit with a threshold value and assesses the fall risk of the managed target person.

### Advantageous Effects of Invention

According to the fall risk assessment system of the present invention, it is possible to easily assess the fall risk of a managed target person such as an elderly person on behalf of a physiotherapist or the like on the basis of photographed images of daily life taken by a stereo camera.

### BRIEF DESCRIPTION OF DRAWINGS

FIG. 1 is a view showing a configuration example of a fall risk assessment system according to a first embodiment.

FIG. 2 is a view showing a detailed configuration example of a 1A section of FIG. 1.

FIG. 3 is a view showing a detailed configuration example of a 1B section of FIG. 1.

FIG. 4A is a view showing an integration unit function.

FIG. 4B is a view showing an integrated data example of the first embodiment.

FIG. 5 is a view showing a detailed configuration example of a fall index calculation unit.

FIG. 6 is a view showing a configuration example of a fall risk assessment system according to a second embodiment.

FIG. 7A is a view showing first half processing of a fall risk assessment system according to a third embodiment.

FIG. 7B is a view showing an integrated data example of the third embodiment.

FIG. 8 is a view showing second half processing of the fall risk assessment system according to the third embodiment.

### DESCRIPTION OF EMBODIMENTS

Hereinafter, embodiments of a fall risk assessment system of the present invention will be described in detail with reference to the drawings. Incidentally, in the following, description will be made as to an example in which an elderly person deteriorated in walking function is targeted for management. However, an injured person or a disabled person or the like who has a high risk of falling may be targeted for management.

### First Embodiment

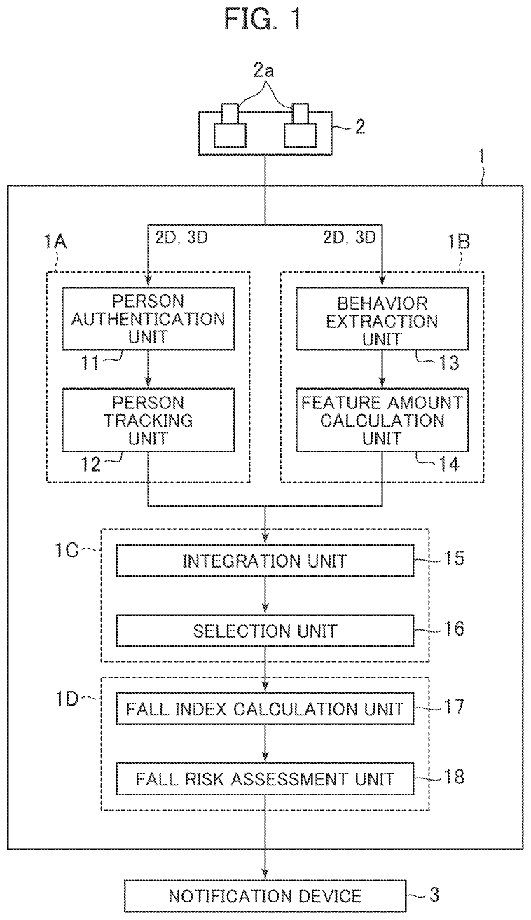

FIG. 1 is a view showing a configuration example of a fall risk assessment system according to a first embodiment of the present invention. This system assesses the fall risk of the elderly to be managed in real time, and is comprised of a fall risk assessment device 1 which is a main part of the present invention, a stereo camera 2 installed in a daily living environment such as a group home, and a notification device 3 such as a display installed in a waiting room or the like for a physiotherapist or a caregiver.

The stereo camera 2 is a camera having a pair of monocular cameras 2a incorporated therein, and simultaneously captures a two-dimensional image 2D from each of the left and right viewpoints to generate three-dimensional information 3D including a depth distance. Incidentally, a method for generating three-dimensional information 3D from a pair of two-dimensional images 2D will be described later.

The fall risk assessment device 1 is a device which assesses the fall risk of the elderly or predicts the fall of the elderly on the basis of the two-dimensional images 2D and the three-dimensional information 3D acquired from the stereo camera 2, and outputs the result of its assessment and the result of its prediction to the notification device 3. Specifically, the fall risk assessment device 1 is a computer such as a personal computer equipped with hardware such as a computing device such as a CPU, a main storage device such as a semiconductor memory, an auxiliary storage device such as a hard disk, and a communication device. Then, each function to be described later is realized by an arithmetic unit executing a program loaded from the auxiliary storage device to the main storage device. In the following, however, such well-known techniques in the computer field will be described while omitting the same as appropriate.

The notification device 3 is a display or a speaker which notifies the output of the fall risk assessment device 1. Information notified here is the name of the elderly person assessed by the fall risk assessment device 1, a face photograph, a change over time in the fall risk, a fall prediction warning, etc. Thus, since the physiotherapist or the like can know the magnitude of the fall risk for each elderly person and its change with time through the notification device 3 without constantly visually observing the elderly person, the burden on the physiotherapist or the like is greatly reduced.

### <Fall Risk Assessment Device 1>

Hereinafter, the fall risk assessment device 1 which is a main part of the present invention will be described in detail. As shown in FIG. 1, the fall risk assessment device 1 includes a person authentication unit 11, a person tracking unit 12, a behavior extraction unit 13, a feature amount calculation unit 14, an integration unit 15, a selection unit 16, a fall index calculation unit 17, and a fall risk assessment unit 18. In the following, each part will be outlined individually, and then cooperative processing of each part will be described in detail.

### <Person Authentication Unit 11>

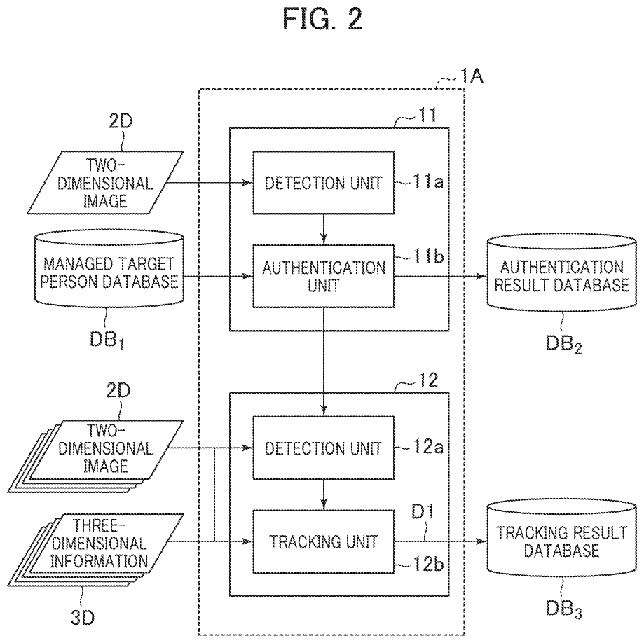

In a daily living environment such as a group home, there may be multiple elderly people, and there may also be caregivers, visitors, etc. who care for the elderly people. Therefore, the person authentication unit 11 utilizes a managed target person database DB₁ (refer to FIG. 2) to identify whether the person captured by the two-dimensional image 2D of the stereo camera 2 is a managed target person. For example, when the person photographed in the two-dimensional image 2D is authenticated as the elderly person being the managed target person in the case where the face reflected in the two-dimensional image 2D and the face photograph registered in the managed target person database DB₁ match each other, etc., the ID and the like of the elderly person are read from the managed target person database DB₁, and the ID and the like are recorded in an authentication result database DB₂ (refer to FIG. 2). Incidentally, the information recorded in the authentication result database DB₂ in association with the ID is, for example, the name, gender, age, face photograph, caregiver in charge, fall history, medical information, and the like of the elderly.

### <Person Tracking Unit 12>

The person tracking unit 12 executes tracking of the target person who wants to evaluate the fall risk, which is authenticated by the person authentication unit 11, by using the two-dimensional image 2D and the three-dimensional information 3D. Incidentally, when the processing capacity of the arithmetic unit is high, all the persons authenticated by the person authentication unit 11 may be persons to be tracked by the person tracking unit 12.

### <Behavior Extraction Unit 13>

After recognizing the behavior type of the elderly person, the behavior extraction unit 13 extracts the behavior related to the fall. For example, it extracts “walking” that is most relevant to falls. The behavior extraction unit 13 can utilize a deep learning technology. For example, using a CNN (Convolutional Neural Network) or an LSTM (Long Short-Term Memory), the behavior extraction unit 13 recognizes the behavior type such as “seating”, “upright”, “walking”, and “falling”, and then extracts the “walking” from among them. There are used for behavior recognition, for example, technologies described in Zhenzhong Lan, Yi Zhu, Alexander G. Hauptmann, “Deep Local Video Feature for Action Recognition”, CVPR, 2017., and Wentao Zhu, Cuiling Lan, Junliang Xing, Wenjun Zeng, Yanghao Li, Li Shen, Xiaohui Xie, “Co-occurrence Feature Learning for Skeleton based Action Recognition using Regularized Deep LSTM Networks”, AAAI 2016.

### <Feature Amount Calculation Unit 14>

The feature amount calculation unit 14 calculates a feature amount from the behavior of each elderly person extracted by the behavior extraction unit 13. For example, when extracting the “walking” behavior, the feature amount calculation unit 14 calculates a feature amount of “walking”. For the calculation of the walking feature amount, there is used, for example, a technology described in Y. Li, P. Zhang, Y. Zhang and K. Miyazaki, “Gait Analysis Using Stereo Camera in Daily Environment,” 2019 41st Annual International Conference of the IEEE Engineering in Medicine and Biology Society (EMBC), Berlin, Germany, 2019, pp. 1471-1475.

### <Integration Unit 15>

The integration unit 15 integrates the output of the feature amount calculation unit 14 from the person authentication unit 11 for each shooting frame of the stereo camera 2, and generates integrated data CD in which the ID and the feature amount or the like are associated with each other. The details of the integrated data CD generated here will be described later.

### <Selection Unit 16>

The two-dimensional image 2D also includes a frame mixed with disturbance such as temporary hiding of the face of an elderly person. When such a frame with the disturbance is processed, the person authentication unit 11 fails in the person authentication, and the person tracking unit 12 fails in the person tracking. In such a case, the integration unit 15 has the possibility of generating integrated data CD low in reliability. For example, when a momentary failure in person authentication occurs, the original ID (for example, ID=1) is momentarily replaced with another ID (for example, ID=2), so that an integrated data CD group discontinuous in ID is generated in the integration unit 15.

Further, since it is necessary to use an integrated data CD group of at least about 20 frames to accurately calculate the feature amount, it is desirable to exclude an integrated data CD group with a short “walking” period of less than 20 frames to correctly calculate the walking feature amount.

When defective data including the above-mentioned ID discontinuity and insufficiency of the “walking” period, and the like is used for subsequent processing, the reliability of the fall risk assessment deteriorates. Therefore, the selection unit 16 assesses the reliability of the integrated data CD, and selects only the highly reliable integrated data CD and outputs the same to the fall index calculation unit 17. Consequently, the selection unit 16 enhances the reliability of the subsequent processing.

### <Fall Index Calculation Unit 17>

The fall index calculation unit 17 calculates a fall index value indicative of the fall risk of the elderly person on the basis of the feature amount of the integrated data CD selected by the selection unit 16.

There are various fall index values. For example, there is a TUG (Timed up and go) score, which is an index value often used for fall assessment. This TUG score is an index value obtained by measuring the time it takes for an elderly person to get up from a chair, walk, and then sit down again. The TUG score is taken to be an index value that has a strong correlation with high and low walking functions. If the TUG score is 13.5 seconds or more, it can be determined that the risk of falling is high. The details of the TUG score have been described in, for example, “Predicting the probability for falls in community-dwelling older adults using the Timed Up & Go Test” by Shumway-Cook A, Brauer S, Woollacott M., Physical Therapy. Volume 80. Number 9. September 2000, pp. 896-903.

When the TUG score is adopted as the fall index value, the fall index calculation unit 17 extracts the behavior for each elderly person from the integrated data CD of each frame, counts a behavior time required to complete a series of movements in the order of (1) sit down, (2) stand upright (or walk), and (3) sit down, and calculates the counted number of seconds as a TUG score. Incidentally, the details of a method for calculating the TUG score have been described in, for example, “Gait Analysis Using Stereo Camera in Daily Environment,” by Y. Li, P. Zhang, Y. Zhang and K. Miyazaki, 2019 41st Annual International Conference of the IEEE Engineering in Medicine and Biology Society (EMBC), Berlin, Germany, 2019, pp. 1471-1475.

Also, the fall index calculation unit 17 may construct a TUG score calculation model from the accumulated elderly data using a machine learning SVM (support vector machine) and estimate a daily TUG score for the elderly using the calculation model. Further, the fall index calculation unit 17 can construct an estimation model of the TUG score from the accumulated elderly data even by using deep learning. Incidentally, the calculation model and the estimation model may be constructed for each elderly person.

### <Fall Risk Assessment Unit 18>

The fall risk assessment unit 18 assesses the fall risk on the basis of the fall index value (for example, TUG score) calculated by the fall index calculation unit 17. Then, when the risk of falling is high, an alarm is issued to a physiotherapist, a caregiver, or the like via the notification device 3.

### <Cooperative Processing in 1A Section of FIG. 1>

Next, the details of cooperative processing between the person authentication unit 11 and the person tracking unit 12 shown in a 1A section of FIG. 1 will be described using FIG. 2.

The person authentication unit 11 authenticates whether an elderly person reflected in the two-dimensional image 2D is a managed target person, and has a detection unit 11a and an authentication unit 11b.

The detection unit 11a detects the face of the elderly person reflected in the two-dimensional image 2D. As a face detection method, various methods such as a conventional matching method and a recent deep learning technique can be utilized, and the present invention does not limit this method.

The authentication unit 11b collates the face of the elderly person detected by the detection unit 11a with the face photograph registered in the managed target person database DB₁. When the face matches with the face photograph, the authentication unit 11b identifies the ID of the authenticated elderly person. When the ID does not exist in the managed target person database DB₁, a new ID is registered as needed. This authentication processing may be performed on all frames of the two-dimensional image 2D, but in the case where the processing speed of the arithmetic unit is low, etc., the authentication processing is performed only on a frame in which an elderly person first appears or reappears. After that, the authentication processing may be omitted.

On the other hand, the person tracking unit 12 monitors the trajectories of movement of the elderly person authenticated by the person authentication unit 11 in time series, and has a detection unit 12a and a tracking unit 12b.

The detection unit 12a detects a body area of the elderly person to be monitored from a plurality of continuous two-dimensional images 2D and three-dimensional information 3D, and further creates a frame indicating the body area. Incidentally, in FIG. 2, the detection unit 11a which detects the face, and the detection unit 12a which detects the body area are separately provided, but one detection unit may detect both the face and the body area.

The tracking unit 12b determines whether or not the same elderly person is detected by a plurality of continuous two-dimensional images 2D and three-dimensional information 3D. In tracking, a person is first detected on a two-dimensional image 2D, and its continuity is determined to perform tracking. Here, the tracking on the two-dimensional image 2D has an error. For example, when different people exist nearby, or they cross each other and walk, the tracking may be wrong. Therefore, for example, the three-dimensional information 3D is utilized to determine the position of a person, the walking direction thereof, and the like, so that the tracking can be performed correctly. Then, when the same elderly person is determined to have been detected, the tracking unit 12b stores the movement locus of the frame indicating the body area of the elderly person in the tracking result database DB₃ as tracking result data D₁. The tracking result data D₁ may include a series of images of the elderly person.

Incidentally, when there is a frame in which the person authentication unit 11 fails in authentication but the person tracking unit 12 succeeds in tracking, the elderly person reflected in the frame may be authenticated as the same person as the elderly person reflected in the previous and following frames. Further, when each of frames in which the elderly person could not be detected is mixed in each continuous frame of the two-dimensional image 2D, the movement locus of the elderly person in the frame may be complemented based on the position of the elderly person detected in the frames before and after the frame.

### <Cooperative Processing in 1B Section of FIG. 1>

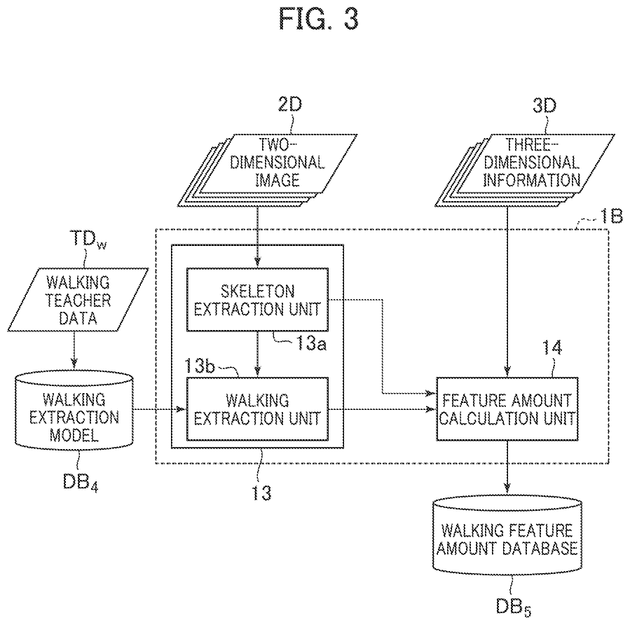

Next, the details of the cooperative processing between the behavior extraction unit 13 and the feature amount calculation unit 14 shown in a 1B section of FIG. 1 will be described using FIG. 3. This will be described as a “walking” behavior most closely related to falls.

The behavior extraction unit 13 recognizes the behavior type of the elderly and then extracts “walking” from among them. The behavior extraction unit 13 has a skeleton extraction unit 13a and a walking extraction unit 13b.

First, the skeleton extraction unit 13a extracts skeleton information of the elderly from the two-dimensional image 2D.

Then, the walking extraction unit 13b extracts “walking” from various behaviors of the elderly by using a walking extraction model DB₄ learned by the walking teacher data TD_{w} and the skeleton information extracted by the skeleton extraction unit 13a. Since the form of “walking” may differ greatly for each elderly person, it is desirable to use the walking extraction model DB₄ according to the condition of the elderly. For example, when targeting elderly people undergoing knee rehabilitation, “walking” is extracted using the walking extraction model DB₄ characterized by knee bending. Other “walking” modes can also be added as needed. Incidentally, although not shown, the behavior extraction unit 13 includes a seating extraction unit, an upright extraction unit, a fall extraction unit, and the like even in addition to the walking extraction unit 13b, and can extract the behaviors such as “seating”, “upright”, and “falling”.

When “walking” is extracted by the walking extraction unit 13b, the feature amount calculation unit 14 calculates a feature amount of the walking. This walking feature amount is the walking speed Speed, walking stride length, etc. of the elderly person to be monitored, which are calculated using the skeletal information and three-dimensional information 3D. The calculated walking feature amount is stored in the walking feature amount database DB₅.

Next, the details of a method of generating the three-dimensional information 3D from the pair of left and right two-dimensional images 2D by the stereo camera 2 will be described.

An equation 1 is an internal parameter matrix K of the stereo camera 2, and an equation 2 is a calculation equation of an external parameter matrix D of the stereo camera 2.

$$K = [ \begin{matrix}f & sf & {u}_{c} & 0 \\ 0 & af & {v}_{c} & 0 \\ 0 & 0 & 1 & 0\end{matrix} ] \qquad ( Equation 1 )$$

$$D = [ \begin{matrix}{r}_{11} & {r}_{12} & {r}_{13} & {t}_{X} \\ {r}_{21} & {r}_{22} & {r}_{23} & {t}_{Y} \\ {r}_{31} & {r}_{32} & {r}_{33} & {t}_{Z} \\ 0 & 0 & 0 & 1\end{matrix} ] \qquad ( Equation 2 )$$

Here, f in the equation 1 indicates a focal length, a_{f} indicates an aspect ratio, s_{f} indicates skew, and (v_{c}, u_{c}) indicates the center coordinates of image coordinates. Further, (r₁₁, r₁₂, r₁₃, r₂₁, r₂₂, r₂₃, r₃₁, r₃₂, r₃₃) in the equation 2 indicates the orientation of the stereo camera 2, and (t_{X}, t_{Y}, t_{Z}) indicates the world coordinates of the installation position of the stereo camera 2.

Using these two parameter matrices K and D and a constant λ, the image coordinates (u, v) and the world coordinates (X, Y, Z) can be associated with each other by the relational expression of an equation 3.

$$λ [ \begin{matrix}u \\ v \\ 1\end{matrix} ] = KD [ \begin{matrix}X \\ Y \\ Z \\ 1\end{matrix} ] \qquad ( Equation 3 )$$

Incidentally, when (r₁₁, . . . , r₃₃) in the equation 2, which indicates the orientation of the stereo camera 2, is defined by Euler angles, it is represented by three parameters of pan θ, tilt ϕ, and roll φ which are the installation angles of the stereo camera 2. Therefore, the number of camera parameters required for associating the image coordinates with the world coordinates becomes 11, which is the total of five internal parameters and six external parameters. Distortion correction and parallelization processing are performed using these parameters.

In the stereo camera 2, three-dimensional measured values of a measured object are calculated by equations 4 and 5.

$$( \begin{matrix}{u}_{l} \\ {v}_{l}\end{matrix} ) = \frac{f}{Z} ( \begin{matrix}X + \frac{B}{2} \\ Y\end{matrix} ) \qquad ( Equation 4 )$$

$$( \begin{matrix}{u}_{r} \\ {v}_{r}\end{matrix} ) = \frac{f}{Z} ( \begin{matrix}X - \frac{B}{2} \\ Y\end{matrix} ) \qquad ( Equation 5 )$$

(uₗ, vₗ) in the equation 4 and (uᵣ, vᵣ) in the equation 5 are respectively pixel values on the left and right two-dimensional images 2D captured by the stereo camera 2. After the parallelization processing, vₗ=vᵣ=v. Incidentally, in both equations, f is the focal length and B is the distance (baseline) between the monocular cameras 2a.

Further, the equations 4 and 5 are arranged using a parallax d. Incidentally, the parallax d is a difference between images obtained by projecting the same three-dimensional measured object onto the left and right monocular cameras 2a. The relationship between the world coordinates and the image coordinates expressed using the parallax d is as shown in an equation 6.

$$( \begin{matrix}X \\ Y \\ Z\end{matrix} ) = \frac{B}{d} ( \begin{matrix}( {u}_{l} + {u}_{r} ) / 2 \\ v \\ f\end{matrix} ) \qquad ( Equation 6 )$$

In the stereo camera 2, the three-dimensional information 3D is generated from the pair of two-dimensional images 2D according to the above processing flow.

Returning to FIG. 3, the description of the skeleton extraction unit 13a and the feature amount calculation unit 14 will be continued.

The skeleton extraction unit 13a extracts the skeleton of the elderly person from the two-dimensional image 2D. It is better to use the Mask R-CNN method in order to extract the skeleton. Mask R-CNN can utilize software “Detectron” or the like, for example (Detectron. Ross Girshick, Ilija Radosavovic, Georgia Gkioxari, Piotr Doll, Kaiming He. https://github.com/facebookresearch/detectron. 2018.)

According to this, first, 17 nodes of a person are extracted. The 17 nodes are the head, left eye, right eye, left ear, right ear, left shoulder, right shoulder, left elbow, right elbow, left wrist, right wrist, left waist, right waist, left knee, right knee, left ankle, and right ankle. Using the center coordinates of the image coordinates (v_{c}, u_{c}), image information feature_{2D} of the 17 nodes by the two-dimensional image 2D can be expressed by an equation 7.

feature_{2D}ⁱ={[u₁, v₁], . . . , [u₁₇, v₁₇]} (Equation 7)

The equation 7 is equivalent to a mathematical expression of the characteristics of the 17 nodes in image coordinates. This is converted as world coordinate information for the same nodes by an equation 8 to obtain 17 three-dimensional information 3Ds. Incidentally, a stereo method or the like can be used to calculate the three-dimensional information.

feature_{3D}ⁱ={[x₁, y₁z₁], . . . , [x₁₇, y₁₇, z₁₇]}. . . (Equation 8)

Next, the feature amount calculation unit 14 calculates the center point (v₁₈, u₁₈) of the 17 nodes using equations 9 and 10. Incidentally, three-dimensional information corresponding to the center point (v₁₈, u₁₈) is assumed to be (X₁₈, Y₁₈, Z₁₈).

$${v}_{18} = \frac{[ max ( {v}_{1} , … , {v}_{17} ) + min ( {v}_{1} , … , {v}_{17} ) ]}{2} \qquad ( Equation 9 )$$

$${u}_{18} = \frac{[ max ( {u}_{1} , … , {u}_{17} ) + min ( {u}_{1} , … , {u}_{17} ) ]}{2} \qquad ( Equation 10 )$$

Next, the feature amount calculation unit 14 calculates a walking speed Speed by an equation 11 using the displacement of the three-dimensional information of a total of 18 points comprised of the 17 nodes and the center point within a predetermined time. Here, the predetermined time t₀ is, for example, 1.5 seconds.

$$Speed = {\sum }_{i = 1}^{18} \frac{\sqrt{{( {x}_{i}^{t - {t}_{0}} - {x}_{i}^{t} )}^{2} + {( {y}_{i}^{t - {t}_{0}} - {y}_{i}^{t} )}^{2} + {( {z}_{i}^{t - {t}_{0}} - {z}_{i}^{t} )}^{2}}}{18 * {t}_{0}} \qquad ( Equation 11 )$$

Further, the feature amount calculation unit 14 uses the three-dimensional information (x₁₆, y₁₆, z₁₆) and (x₁₇, y₁₇, z₁₇) of the nodes of the left and right ankles in each frame to calculate a distance dis between the left and right ankles in each frame by an equation 12.

dis=\sqrt{(x₁₆−x₁₇)²+(y₁₆−y₁₇)²+(z₁₆−z₁₇)²} (Equation 12)

Then, the feature amount calculation unit 14 calculates a stride length on the basis of the distance dis calculated for each frame. Here, as shown in an equation 13, the largest distance dis calculated in a predetermined time zone is calculated as the stride length. When the predetermined time is set to 1.0 second, the maximum value of the distance dis calculated from each of the plurality of frames taken during that period is extracted and taken as the stride length.

length=max{disₜ₋ₙ, . . . , disₜ₋₁,disₜ} (Equation 13)

The feature amount calculation unit 14 further calculates a necessary walking feature amount such as acceleration by using the walking speed Speed and the stride length. The details of a method of calculating these feature amounts have been described in, for example, the paper “Identification of fall risk predictors in daily life measurements: gait characteristics' reliability and association with self-reported fall history”, by Rispens S M, van Schooten K S, Pijnappels M et al., Neurorehabilitation and neural repair, 29 (1):54-61, 2015.

Through the above processing, the feature amount calculation unit 14 calculates a plurality of walking feature amounts (walking speed, stride, acceleration, etc.) and registers them in the walking feature amount database DB₅.

### <Cooperative Processing in 1C Section of FIG. 1>

Next, the details of the cooperative processing between the integration unit 15 and the selection unit 16 shown in the 1C section of FIG. 1 will be described.

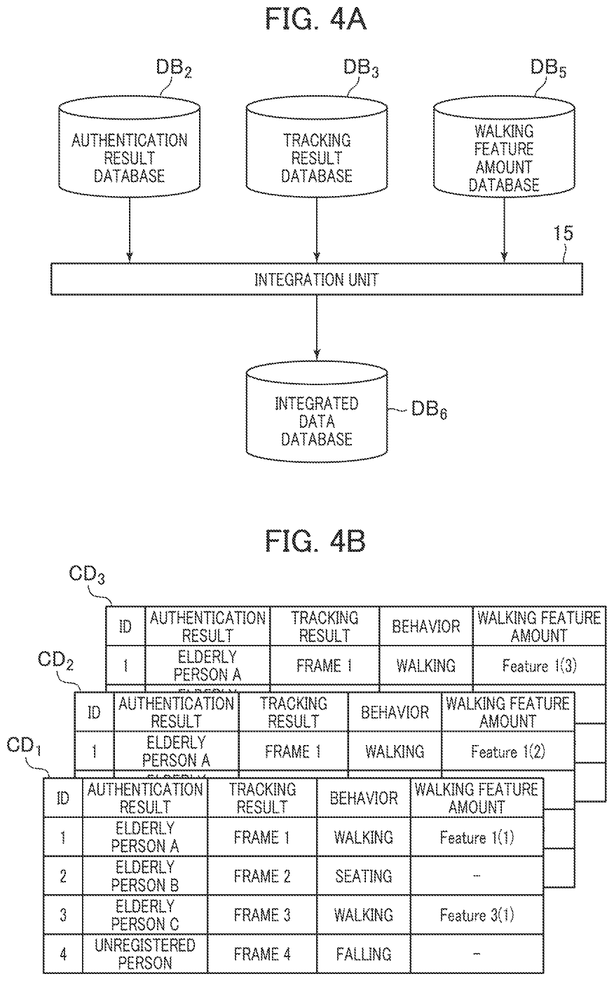

First, the processing in the integration unit 15 will be described using FIG. 4A. As shown herein, the integration unit 15 integrates the data registered in the authentication result database DB₂, the tracking result database DB₃, and the walking feature amount database DB₅ for each shooting frame of the stereo camera 2 to generate integrated data CD. Then, the integration unit 15 registers the generated integrated data CD in the integrated data database DB₆.

As shown in FIG. 4B, the integrated data CDs (CD₁ to CDₙ) of each frame are tabular data obtained by summarizing for each ID, authentication results (names of elderly people, etc.), tracking results (corresponding frames), behavior contents, and walking feature amounts (walking speed, etc.) when the behavior contents are “walking”. Incidentally, when an unregistered person is detected, a new ID (ID=4 in the example of FIG. 4B) may be assigned to that person and various related information may be integrated. If reference is sequentially made to such a series of integrated data CDs, it is possible to continuously detect the walking feature amount of each elderly person to be managed taken by the stereo camera 2.

The selection unit 16 selects data having met the criterion from the integrated data CD integrated by the integrated unit 15 and outputs the same to the fall index calculation unit 17. The selection criterion in the selection unit 16 can be set according to the installation location of the stereo camera 2 and the behavior of the elderly person. For example, when the behavior of the same elderly person is recognized as “walking” continuously for 20 frames or more, it is conceivable to select and output a series of walking feature amounts thereof.

### <Cooperative Processing in 1D Section of FIG. 1>

Next, the details of the cooperative processing between the fall index calculation unit 17 and the fall risk assessment unit 18 shown in a 1D section of FIG. 1 will be described.

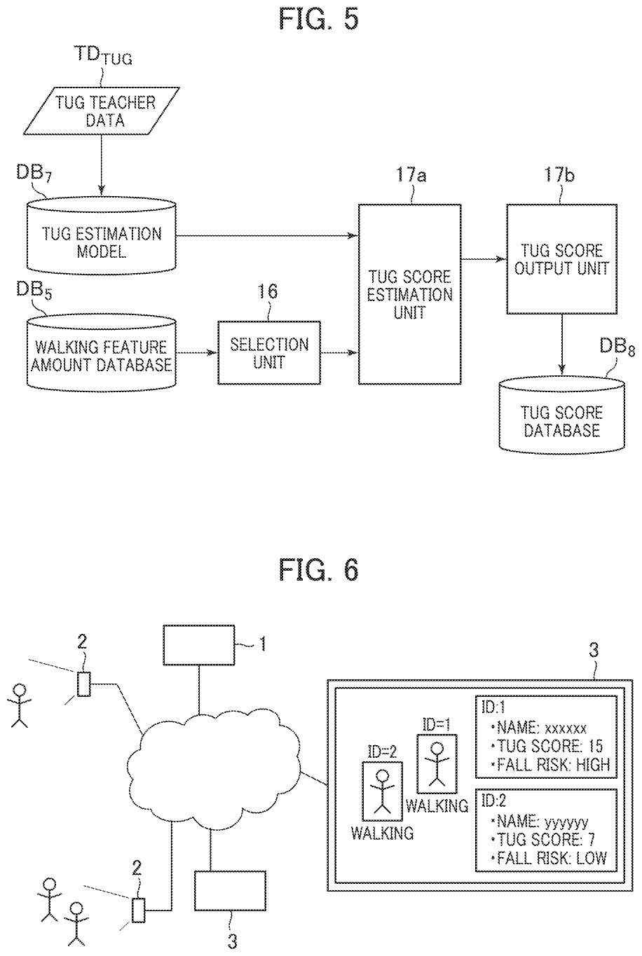

First, the fall index calculation unit 17 will be described using FIG. 5. There are various fall indexes used for assessing the fall risk, but in the present embodiment in which the TUG score is adopted as the fall index, the fall index calculation unit 17 has a TUG score estimation unit 17a and a TUG score output unit 17b.

A TUG estimation model DB₇ is an estimation model used to estimate the TUG score, based on the walking feature amount, and is learned in advance from TUG teacher data TD_{TUG}, which is a set of the walking feature amount and the TUG score.

The TUG score estimation unit 17a estimates the TUG score by using the TUG estimation model DB₇ and the walking feature amount selected by the selection unit 16. Then, the TUG score output unit 17b registers the TUG score estimated by the TUG score estimation unit 17a in a TUG score database DB₈ in association with the ID.

The fall risk assessment unit 18 assesses the fall risk on the basis of the TUG score registered in the TUG score database DB₈. As described above, when the TUG score is 13.5 seconds or more, it can be determined that the fall risk is high. Therefore, when this is the case, the fall risk assessment unit 18 issues warning to the physiotherapist or caregiver or the like in charge via the notification device 3. As a result, the physiotherapist, caregiver or the like may rush under the elderly person high in fall risk to assist in walking, or change the services provided to the elderly person in the future to be more generous.

According to the fall risk assessment system of the present embodiment described above, the fall risk of the person to be managed such as the elderly person can be easily assessed instead of the physiotherapist, etc., based on the images of daily life taken by the stereo camera.

### Second Embodiment

Next, a fall risk assessment system according to a second embodiment of the present invention will be described using FIG. 6. It is noted that as for the common points with the first embodiment, dual explanations will be omitted.

The fall risk assessment system of the first embodiment is a system in which one stereo camera 2 and one notification device 3 are directly connected to the fall risk assessment device 1, and is a system suitable for use in small-scales facilities.

On the other hand, in a large-scale facility, it is convenient if a large number of elderly people photographed by stereo cameras 2 installed in various places can be unitarily managed. Therefore, in the fall risk assessment system of the present embodiment, a plurality of stereo cameras 2 and notification devices 3 are connected to one fall risk assessment device 1 through a network such as a LAN (Local Area Network), cloud, wireless communication, or the like. This enables remote management of a large number of elderly people in various locations. For example, in a four-story long-term care facility, a stereo camera 2 can be installed on each floor to assess the fall risk of elderly people on each floor from one place. Further, the notification device 3 does not need to be installed in the facility where the stereo camera 2 is installed, and the notification device 3 installed in a remote management center or the like may manage a large number of elderly people in the nursing facilities.

An example of the display screen of the notification device 3 is shown on the right side of FIG. 6. Here, the “ID”, “frame showing the body area”, and “behavior” are displayed with superposed on the image of the elderly person reflected in the two-dimensional image 2D. Further, in the right window, the name of each elderly person, TUG score, and the magnitude of fall risk are displayed. The change in TUG score over time may be displayed in this window.

According to the fall risk assessment system of the present embodiment described above, it is possible to easily assess the fall risk of a large number of elderly people in various places even when a large-scale facility is to be managed.

### Third Embodiment

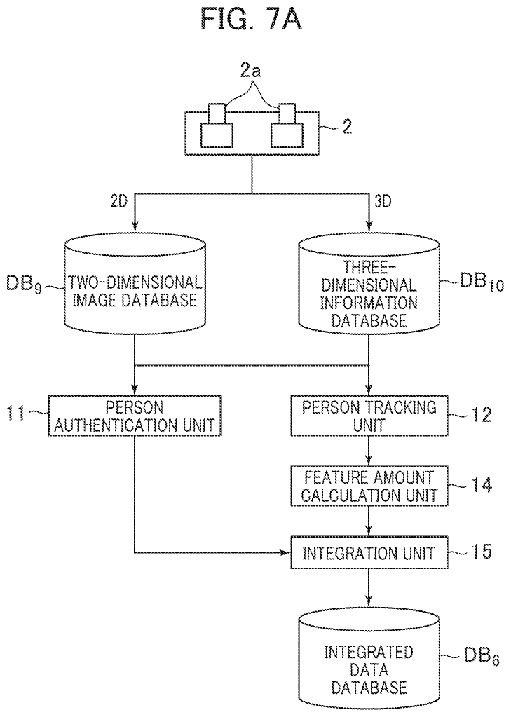

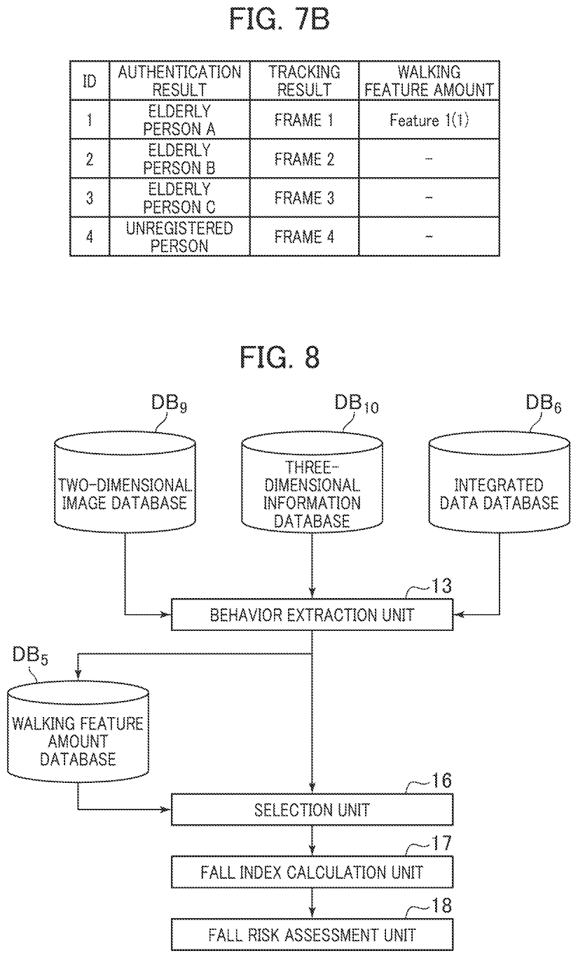

Next, a fall risk assessment system according to a third embodiment of the present invention will be described using FIGS. 7A to 8. It is noted that as for the common points with the above embodiment, dual explanations will be omitted.

Since the fall risk assessment system of each of the first and second embodiments is a system which assesses the fall risk of the managed target person in real time, it is necessary to constantly start and always connect the fall risk assessment device 1 and the stereo camera 2.

On the other hand, the fall risk assessment system of the present embodiment is a system in which normally only the stereo camera 2 is started, and the fall risk assessment device 1 is started as needed to thereby enable the fall risk of an elderly person to be assessed ex post. Therefore, the system of the present embodiment is a system in which if it not only does not require constant connection between the fall risk assessment device 1 and the stereo camera 2 and constant activation of the fall risk assessment device 1, but also includes a storage medium to and from which the stereo camera 2 can be attached and detached, the shooting data of the stereo camera 2 can be input to the fall risk assessment device 1 without connecting the fall risk assessment device 1 and the stereo camera 2 at all.

FIG. 7A is a view outlining the first half processing of the fall risk assessment system of the present embodiment. In the present embodiment, first, a two-dimensional image 2D output by the stereo camera 2 is stored in a two-dimensional image database DB₉, and three-dimensional information 3D is stored in a three-dimensional information database DB₁₀. These databases are recorded in, for example, a recording medium such as a detachable semiconductor memory card. Incidentally, the two-dimensional image database DB₉ and the three-dimensional information database DB₁₀ may store all the data output by the stereo camera 2, but when the recording capacity of the recording medium is small, only data with a person being detected through a background difference method or the like may be extracted and stored therein.

When a sufficient amount of data is accumulated in both databases, the assessment processing of the fall risk by the fall risk assessment device 1 can be started.

As shown in FIG. 7A, in the fall risk assessment device 1 of the present embodiment, since the behavior extraction unit 13 is not provided in the preceding stage of the integration unit 15, the feature amount calculation unit 14 calculates walking feature amounts for all the behaviors of the elderly. Thus, unlike the first embodiment, the integrated data CD of the present embodiment generated by the integration unit 15 does not have data indicating the behavior type, but when there is actually “walking”, the walking feature amount is recorded (refer to FIG. 7B).

FIG. 8 is a view outlining the second half processing of the fall risk assessment system of the present embodiment. When all of the three types of databases shown in FIG. 7A are generated, the behavior extraction unit 13 of the fall risk assessment device 1 refers to the column of the walking feature amount of the integrated data CD illustrated in FIG. 7B to extract “walking”. Then, by executing the processing similar to that in the first embodiment, the fall risk of the elderly person is assessed ex post.

According to the fall risk assessment system of the present embodiment described above, since it is not necessary to constantly start and always connect the fall risk assessment device 1 and the stereo camera 2, not only can the power consumption amount of the fall risk assessment device 1 be reduced, but also the fall risk assessment device 1 and the stereo camera 2 need not be connected at all if the stereo camera 2 is provided with the detachable storage medium. Therefore, in the system of the present embodiment, there is no need to consider the connection of the stereo camera 2 to the network, so that the stereo camera 2 can be freely installed in various places.

### LIST OF REFERENCE SIGNS

1 . . . fall risk assessment device, 11 . . . person authentication unit, 11a . . . detection unit, 11b . . . authentication unit, 12 . . . person tracking unit, 12a . . . detection unit, 12b . . . tracking unit, 13 . . . behavior extraction unit, 13 a . . . skeleton extraction unit, 13b . . . walking extraction unit, 14 . . . feature amount calculation unit, 15 . . . integration unit, 16 . . . selection unit, 17 . . . fall index calculation unit, 17a . . . TUG score estimation unit, 17b . . . TUG score output unit, 18 . . . fall risk assessment unit, 2 . . . stereo camera, 2a . . . monocular camera, 3 . . . notification device, 2D . . . two-dimensional image, 3D . . . three-dimensional information, DB₁ . . . managed target person database, DB₂ . . . authentication result database, DB₃ . . . tracking result database, DB₄ . . . walking extraction model, DB₃ . . . walking feature amount database, DB₆ . . . integrated data database, DB₇ . . . TUG estimation model, DB₈ . . . TUG score database, DB₉ . . . two-dimensional image database, DB₁₀ . . . three-dimensional information database, TD_{w} . . . walking teacher data, TD_{TUG} . . . TUG teacher data.

1. A fall risk assessment system, comprising:
a stereo camera which photographs a target person to be managed and outputs a two-dimensional image and three-dimensional information; and
a fall risk assessment device which assesses the fall risk of the managed target person,
wherein the fall risk assessment device includes:
a person authentication unit which authenticates the managed target person photographed by the stereo camera,
a person tracking unit which tracks the managed target person authenticated by the person authentication unit,
a behavior extraction unit which extracts the walking of the managed target person,
a feature amount calculation unit which calculates a feature amount of the walking extracted by the behavior extraction unit,
an integration unit which generates integrated data which integrates the outputs of the person authentication unit, the person tracking unit, the behavior extraction unit, and the feature amount calculation unit,
a fall index calculation unit which calculates a fall index value of the managed target person, based on a plurality of the integrated data generated by the integration unit, and
a fall risk assessment unit which compares the fall index value calculated by the fall index calculation unit with a threshold value and assesses the fall risk of the managed target person.

2. The fall risk assessment system according to claim 1, wherein the fall risk assessment device further includes a selection unit which selects highly reliable data from the plurality of integrated data generated by the integration unit.

3. The fall risk assessment system according to claim 2, wherein among the plurality of integrated data generated by the integration unit, the selection unit outputs an integrated data group in which the behavior extracted by the behavior extraction unit is walking continuously for a predetermined number of times or more as highly reliable integrated data.

4. The fall risk assessment system according to claim 3, wherein the fall index calculation unit calculates the fall index value using the walking feature amount selected by the selection unit.

5. The fall risk assessment system according to claim 4, wherein the fall index calculation unit calculates a TUG score as the fall index value, and
wherein when the TUG score is higher than or equal to a threshold value, the fall risk assessment unit determines the fall risk of the managed target person to be high.

6. The fall risk assessment system according to claim 1, wherein when the person authentication unit authenticates a plurality of the managed target persons, the fall index calculation unit calculates the fall index value for each person to be managed, and
the fall risk assessment unit assesses the fall risk for each managed target person.

7. The fall risk assessment system according to claim 1, further including a notification device,
wherein the notification device displays the fall index value or the fall risk for each of the managed target persons authenticated by the person authentication unit.

8. The fall risk assessment system according to claim 1, wherein a plurality of the stereo cameras and the fall risk assessment device are connected via a network.

9. The fall risk assessment system according to claim 1, wherein a facility in which the stereo camera is installed and a facility in which the fall risk assessment device is installed are different.

10. The fall risk assessment system according to claim 1, wherein the stereo camera and the fall risk assessment device are constantly connected, and
wherein the fall risk assessment device assesses the fall risk of the managed target person in real time.

11. The fall risk assessment system according to claim 1, wherein the stereo camera and the fall risk assessment device are not always connected,
wherein the fall risk assessment device assesses the fall risk of the managed target person in an ex-post manner.

12. The fall risk assessment system according to claim 11, wherein the input of data from the stereo camera to the fall risk assessment device is performed via a detachable recording medium.
